(19) United States
(12) Patent Application Publication
(10) Pub. No.: US 2012/0149994 A1
(43) Pub. Date: Jun. 14, 2012

(54) METHOD AND SYSTEM FOR CONTROLLING NON-INVASIVE BLOOD PRESSURE DETERMINATION BASED ON OTHER PHYSIOLOGICAL PARAMETERS

(75) Inventors: William J. Luczyk, Waukesha, WI (US); Lawrence T. Hersh, Milwaukee, WI (US); Robert F. Donehoo, Colgate, WI (US); Bruce A. Friedman, Jasper, GA (US)

(73) Assignee: GENERAL ELECTRIC COMPANY, Schenectady, NY (US)

(21) Appl. No.: 12/967,256

(22) Filed: Dec. 14, 2010

(51) Int. Cl. A61B 5/02; A61B 5/022

(52) U.S. Cl. 600/301; 600/490

(57) ABSTRACT

A system and method for processing a cuff pressure waveform to determine the blood pressure of a patient. The processing unit of the NIBP monitoring system receives status signals from one or more physiological parameter monitors. The physiological parameter monitors each include an operating algorithm that causes the physiological parameter monitor to generate a status signal indicating whether artifacts are present that prevent the determination of the physiological parameter. When the processing unit receives the monitoring signal from the physiological parameter monitor indicating the presence of artifacts, the processing unit adjusts the operation of the NIBP monitor. The adjustment of the NIBP monitor may be to delay the beginning of the NIBP determination cycle until artifacts are no longer present from the physiological parameter monitor or to control the cuff pressure in such a manner as to keep the patient safe and comfortable until the artifacts are no longer present.

### BACKGROUND OF THE INVENTION

The present disclosure generally relates to the field of non-invasive blood pressure (NIBP) monitoring. More specifically, the present disclosure relates to a method and system for monitoring other physiological parameters from a patient to determine whether artifacts are present that may affect the operation of the NIBP monitor in determining the blood pressure of the patient.

The human heart periodically contracts to force blood through the arteries. As a result of this pumping action, pressure pulses or oscillations exist in these arteries and cause them to cyclically change volume. The minimum pressure during each cycle is known as the diastolic pressure and the maximum pressure during each cycle is known as the systolic pressure. A further pressure value, known as the “mean arterial pressure” (MAP) represents a time-weighted average of the measured blood pressure over each cycle.

While many techniques are available for the determination of the diastolic, systolic, and mean arterial pressures of a patient, one such method typically used in non-invasive blood pressure monitoring is referred to as the oscillometric technique. This method of measuring blood pressure involves applying an inflatable cuff around an extremity of a patient's body, such as the patient's upper arm. The cuff is then inflated to a pressure above the patient's systolic pressure and then incrementally reduced in a series of small pressure steps. A pressure sensor pneumatically connected to the cuff measures the cuff pressure throughout the deflation process. The sensitivity of the sensor is such that it is capable of measuring the pressure fluctuations occurring within the cuff due to blood flowing through the patient's arteries. With each beat, blood flow causes small changes in the artery volume which are transferred to the inflated cuff, further causing slight pressure variations within the cuff which are then detected by the pressure sensor. The pressure sensor produces an electrical signal representing the cuff pressure level combined with a series of small periodic pressure variations associated with the beats of a patient's heart for each pressure step during the deflation process. It has been found that these variations, called “complexes” or “oscillations,” have a peak-to-peak amplitude which is minimal for applied cuff pressures above the systolic pressure.

As the cuff pressure is decreased, the oscillation size begins to monotonically grow and eventually reaches a maximum amplitude. After the oscillation size reaches the maximum amplitude, the oscillation size decreases monotonically as the cuff pressure continues to decrease. Oscillometric data such as this is often described as having a “bell curve” appearance. Indeed, a best-fit curve, or envelope, may be calculated representing the amplitude of the measured oscillometric pulses. Physiologically, the cuff pressure at the maximum oscillation amplitude value approximates the MAP. In addition, complex amplitudes at cuff pressures equivalent to the systolic and diastolic pressures have a fixed relationship to this maximum oscillation amplitude value. Thus, the oscillometric method is based upon measurements of detected oscillation amplitudes at various cuff pressures.

Blood pressure measuring devices operating according to the oscillometric method detect the amplitude of the pressure oscillations at various applied cuff pressure levels. The amplitudes of these oscillations, as well as the applied cuff pressure, are stored together as the device automatically changes the cuff pressures through a predetermined pressure pattern. These oscillation amplitudes define an oscillometric “envelope” and are evaluated to find the maximum value and its related cuff pressure, which is approximately equal to MAP. The cuff pressure below the MAP value which produces an oscillation amplitude having a certain fixed relationship to the maximum value is designated as the diastolic pressure, and, likewise, the cuff pressures above the MAP value which results in complexes having an amplitude with a certain fixed relationship to that maximum value is designated as the systolic pressure. The relationships of oscillation amplitude at systolic and diastolic pressures, respectively, to the maximum value at MAP are empirically derived ratios depending on the preferences of those of ordinary skill in the art. Generally, these ratios are designated in the range of 40%-80% of the amplitude at MAP.

One way to determine oscillation magnitudes is to computationally fit a curve to the recorded oscillation amplitudes and corresponding cuff pressure levels. The fitted curve may then be used to compute an approximation of the MAP, systolic and diastolic data points. An estimate of MAP is taken as the cuff pressure level with the maximum oscillation. One possible estimate of MAP may therefore be determined by finding the point on the fitted curve where the first derivative equals zero. From this maximum oscillation value data point, the amplitudes of the oscillations at the systolic and diastolic pressures may be computed by taking a percentage of the oscillation amplitude at MAP. In this manner, the systolic data point and the diastolic data point along the fitted curve may each be computed and therefore their respective pressures may also be estimated. This curve fitting technique has the advantage of filtering or smoothing the raw oscillometric data. However, in some circumstances it has been found that additional filtering techniques used to build and process the oscillometric envelope could improve the accuracy of the determination of the blood pressure values.

The reliability and repeatability of blood pressure computations hinges on the ability to accurately determine the oscillation amplitudes. However, the determination of the oscillation amplitudes is susceptible to artifact contamination. Since the oscillometric method is dependent upon detecting tiny fluctuations in measured cuff pressure, outside forces affecting this measured cuff pressure may produce artifacts that in some cases may completely mask or otherwise render the oscillometric data useless. One such source of artifacts is from voluntary or involuntary motion by the patient. Involuntary movements, such as the patient shivering, may produce high frequency artifacts in the oscillometric data. Voluntary motion artifacts, such as those caused by the patient moving his or her arm, hand, or torso, may produce low frequency artifacts.

During the process of determining the blood pressure of a patient, the algorithm used to calculate the blood pressure, based upon the detected oscillation amplitudes, decides if artifacts are present and if such artifacts make it impossible to detect oscillation amplitudes which have a predicted relationship to amplitudes detected at other pressure steps. When the operating algorithm cannot determine the oscillation amplitude at a particular pressure step, the algorithm may continue to monitor for oscillations at that pressure step. Eventually, after attempting to find oscillations at a pressure step for a sufficiently long time, the algorithm may move on to another step without estimating the oscillation amplitude. In some cases the algorithm may decide to return to the initial inflation pressure and begin the process over. This process of attempting to detect oscillations and possibly moving to other pressure steps continues until the algorithm determines that a blood pressure estimate is impossible to calculate. This delay and uncertainty in processing leads to patient discomfort and possible inaccuracy in the blood pressure estimates.

Various other features, objects and advantages of the invention will be made apparent from the following description taken together with the drawings.

### SUMMARY OF THE INVENTION

A method and system for determining the blood pressure of a patient is disclosed herein. The system includes a processing unit that receives a cuff pressure waveform from a blood pressure cuff applied to the patient. The processing unit receives monitoring signals from one or more physiological parameter monitors and adjusts the operation of the non-invasive blood pressure monitor based upon the monitoring signals from the physiological parameter monitors.

Once the blood pressure cuff has been applied to the patient, the processing unit of the NIBP monitoring system inflates the pressure cuff to an initial inflation pressure. The blood pressure cuff is then deflated in a series of pressure steps from the initial inflation pressure. At each pressure step, the processing unit receives the cuff pressure waveform from the blood pressure cuff. The algorithm contained within the processing unit attempts to determine the size of at least one oscillation amplitude at the pressure step.

If the operating algorithm of the NIBP monitoring system cannot determine the amplitude of the oscillation pulses at the current pressure step, the processing unit checks the monitoring signals from the one or more physiological parameter monitors in communication with the processing unit. The physiological parameter monitors may include an ECG monitor and/or an SpO₂ monitor. Both the ECG monitor and the SpO₂ monitor include operating algorithms that monitor signals from the patient. During the operation of the physiological parameter monitors, the individual monitors determine whether artifacts are present in the signals monitored from the patient. If artifacts are present, the physiological parameter monitor generates a monitoring status signal indicating the presence of the artifacts.

During the process of determining the blood pressure of the patient, the processing unit of the NIBP monitor checks the monitoring status signals from the one or more physiological parameter monitors to determine whether artifacts are present. If the processing unit determines that artifacts are present, the processing unit adjusts the operation of the NIBP monitor.

In one illustrative example, if the processing unit of the NIBP monitoring system determines that artifacts are present in the monitoring status signals from the one or more physiological parameter monitors, the algorithm of the NIBP monitoring system delays the start of the NIBP determination cycle until artifact indications are no longer present in the status signals from the physiological parameter monitors.

In another illustrative example, if the processing unit of the NIBP monitor cannot determine the amplitude of an oscillation pulse at an individual pressure step, the processing unit checks the monitoring status signals from the physiological parameter monitors. If the monitoring status signals indicate that artifacts are present, the processing unit suspends processing the current pressure step, stores the particular pressure level of the step, deflates the cuff pressure, and continues to monitor the signals from the physiological parameter monitors. Once the artifacts are no longer present, the processing unit restarts the monitoring cycle by inflating to the stored pressure level to determine whether the oscillation amplitudes can be measured. Alternatively, the algorithm may decide to start the blood pressure determination over from the initial inflation pressure.

The method and system of the present disclosure utilize monitoring signals from one or more physiological patient monitors to determine whether artifacts are present that may prevent the determination of the blood pressure of the patient. Based upon the monitoring status signals from the physiological parameter monitors, the processing unit of the NIBP monitor adjusts the operation of the NIBP monitor. The adjustment of the NIBP monitor allows the monitor to more accurately calculate the blood pressure of the patient while reducing the periods of cuff pressurization in conditions at which the blood pressure determination is unlikely to be successful.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 depicts an embodiment of a system for the non-invasive measurement of blood pressure;

FIG. 2 is a graph depicting the oscillometric data collected from a blood pressure cuff at multiple pressure steps;

FIG. 3a is a graph illustrating the cuff pressure waveform received from the blood pressure cuff at a cuff pressure step;

FIG. 3b is a graph illustrating the cuff pressure waveform received from the blood pressure cuff at a cuff pressure step where the cuff pressure waveform is corrupted by artifacts;

FIG. 4 is a flowchart depicting the steps carried out by the processing unit of the NIBP monitor; and

FIG. 5 is a flowchart illustrating the blood pressure determination steps carried out by the processing unit.

### DETAILED DESCRIPTION OF THE INVENTION

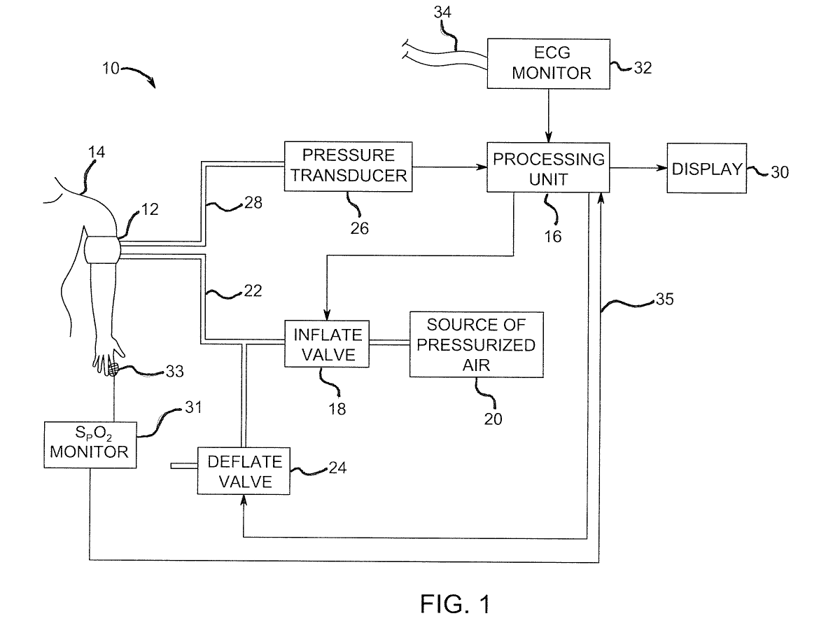

FIG. 1 depicts an embodiment of a non-invasive blood pressure (NIBP) monitoring system 10. The NIBP monitoring system 10 includes a pressure cuff 12 that is a conventional flexible, inflatable and deflatable cuff worn on the arm or other extremity of a patient 14. A processing unit 16 controls an inflate valve 18 that is disposed between a source of pressurized air 20 and a pressure conduit 22. As the inflate valve 18 is controlled to increase the pressure in the cuff 12, the cuff 12 constricts around the arm of the patient 14. Upon reaching a sufficient amount of pressure within the cuff 12, the cuff 12 fully occludes the brachial artery of the patient 14.

After the cuff 12 has been fully inflated, the processing unit 16 further controls a deflate valve 24 to begin incrementally releasing pressure from the cuff 12 back through pressure conduit 22 and out to the ambient air. During the inflation and incremental deflation of the cuff 12, a pressure transducer 26, pneumatically connected to the pressure cuff 12 by pressure conduit 28 measures the pressure within the pressure cuff 12. In an alternative embodiment, the cuff 12 is continuously deflated as opposed to incrementally deflated. In such continuously deflating embodiments, the pressure transducer 26 may measure the pressure within the cuff continuously.

As the pressure within the cuff 12 decreases, the pressure transducer 26 will detect oscillometric pulses in the measured cuff pressure that are representative of the pressure fluctuations caused by the patient's blood flowing into the brachial artery with each heart beat and the resulting expansion of the artery to accommodate the additional volume of blood.

The cuff pressure data as measured by the pressure transducer 26, including the oscillometric pulses, is provided to the processing unit 16 such that the cuff pressure waveform may be processed and analyzed and a determination of the patient's blood pressure, including systolic pressure, diastolic pressure and MAP can be displayed to a clinician on a display 30.

The processing unit 16 may further receive a signal from a physiological parameter monitor, such as the ECG monitor 32. The ECG monitor 32 includes electrical leads 34 connected to specific anatomical locations on the patient 14 monitor the propagation of the electrical activity through the patient's heart. The ECG monitor 32 monitors ECG signals from the patient and calculates various different components and time periods associated with the ECG signal. As an example, the ECG monitor includes internal software that determines QRS complexes and other related time periods. During operation, if the ECG monitor 32 cannot adequately detect various intervals, such as QRS complexes, the ECG monitor 32 includes internal programming that generates a signal indicating that the ECG monitor 32 was unable to perform the required measurements, which may be due to artifacts introduced into the ECG signal.

In the NIBP monitoring system 10 shown in FIG. 1, the system further includes a second physiological parameter monitor, specifically an SpO₂ monitor 31. The SpO₂ monitor 31 includes a finger probe 33 positioned on the patient. The SpO₂ monitor 31 is connected to the NIBP processing unit 16 such that the processing unit 16 receives a signal from the SpO₂ monitor 31. During operation, the SpO₂ monitor 31 includes an operating algorithm that detects the heart rate and blood saturation of the patient 14. If the SpO₂ monitor 31 is unable to generate this desired output signal, the SpO₂ monitor 31 will generate a signal indicating that either artifacts or other sources of corruption prevented the determination of the typical output from the SpO₂ monitor 31. This output is received at the processing unit 16 through the communication line 35.

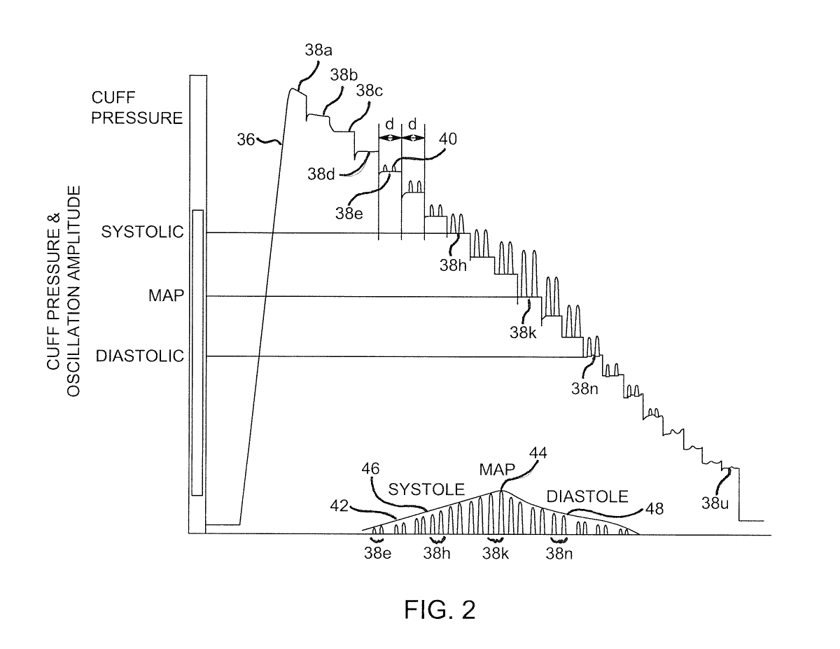

FIG. 2 is a graph depicting various pressure signals that may be acquired from the NIBP monitoring system 10 depicted in FIG. 1. The cuff pressure as determined by the pressure transducer 26 is represented as cuff pressure graph 36. The cuff pressure peaks at the cuff pressure step 38a which is the cuff pressure at which the cuff 12 has been fully inflated as controlled by the processing unit 16. The processing unit 16 controls the inflation of the cuff 12 such that 38a is a pressure that is sufficiently above the systolic pressure of the patient. This may be controlled or modified by referencing previously determined values of patient blood pressure data or by reference to standard medical monitoring practices. The cuff pressure graph 36 then incrementally lowers at a series of pressure steps 38a-38u which reflect each incremental pressure reduction in the cuff 12 as controlled by the deflate valve 24. Before the cuff pressure has reached a pressure step at which the patient brachial artery is no longer completely occluded, the measured cuff pressure will show oscillometric pulses 40. The number of oscillometric pulses detected at each pressure step is controlled as a function of the heart rate of the patient and the length of time that the NIBP system collects data at each pressure step, but typically cuff pressure data is recorded at each pressure level to obtain at least two oscillometric pulses.

The cuff pressure is measured at each of the pressure step increments, including the oscillometric pulse data until the cuff pressure reaches an increment such that the oscillometric pulses are small enough to completely specify the oscillometric envelope, such as found at pressure increment 38u. At this point, the processing unit 16 controls the deflate valve 24 to fully deflate the pressure cuff 12 and the collection of blood pressure data is complete.

FIG. 2 further depicts an oscillometric envelope 42 as calculated using the oscillometric pulse data collected from the series of incremental cuff pressure steps. The processing unit 16 isolates the oscillometric pulses at each pressure step, and creates a best fit curve to represent the oscillometric envelope 42. The oscillometric envelope is useful in estimating systolic pressure, diastolic pressure and MAP. The MAP 44 is determined as the pressure step increment 38k that corresponds to the peak of the oscillometric envelope 42. Once the MAP has been determined, the systolic pressure 46 and diastolic pressure 48 may be identified as the pressure level values associated with particular oscillation amplitudes that are predetermined percentages of the oscillation amplitude at the MAP pressure level. In one embodiment, the systolic pressure 46 corresponds to pressure increment 38h where the oscillometric envelope amplitude is 50% that of the MAP. In another embodiment, the diastolic pressure 48 correlates to pressure increment 38n where the envelope amplitude is between 60% and 70% that of the envelope amplitude at MAP. The percentages of the MAP amplitude used to estimate the systolic pressure and the diastolic pressure are usually between 40% and 80% depending upon the specific algorithm used by the processing unit 16.

In an alternative embodiment, the amplitude of the oscillometric pulses at each pressure step are averaged to produce an oscillometric envelope data point. In some of these embodiments, techniques such as pulse matching or the elimination of the first oscillometric pulse at a pressure step may be used to improve the quality of the computed oscillometric data point. The oscillometric envelope 42 may also be created by using the average of the complex amplitudes at the pressure step as the input data points for a best-fit curve. Alternatively, data points of the oscillometric envelope 42 may be the maximum amplitude of the oscillometric pulses at each pressure step.

As can be seen from FIG. 2, the oscillometric pulses are relatively small with respect to the overall cuff pressure and the pressure increment steps. This makes the detection of the oscillometric pulses highly susceptible to noise and other artifacts. Various filtering techniques can be used to scale and isolate the physiological information in the oscillometric signal for optimal detection of the complexes at each incremental step. However, such filtering techniques are not always able to completely eliminate corrupting artifact.

The physiological monitoring system, and method of determining blood pressure as disclosed herein, aims to provide improved processing of oscillometric pulse signals to respond to the presence of artifacts. FIG. 2 demonstrates an example of acquisition of the oscillometric signals using step deflation; however, other techniques of obtaining the oscillometric signals, such as by continuous deflation, are possible, and the description given here is not meant to limit the usefulness of embodiments as disclosed below with respect to step deflation.

Referring back to FIG. 1, when calculating an automated NIBP measurement in the processing unit 16, it is important to recognize that artifacts can create inaccuracies in the reported blood pressure estimates. In accordance with the present disclosure, the processing unit 16 is connected to at least one physiological parameter monitor, such as the ECG monitor 32 and the SpO₂ monitor 31. Both the ECG monitor 32 and SpO₂ monitor 31 include operating algorithms that are able to determine whether artifacts are detected during the process of obtaining and processing the physiological parameter being monitored. When artifacts are detected, both the ECG monitor 32 and SpO₂ monitor 31 generate a signal indicating the presence of artifacts. The output signals from each of the physiological parameter monitors are received by the processing unit 16 for indicating that artifacts are present. In accordance with the present disclosure, the processing unit 16 receives signals from both the SpO₂ monitor 31 and the ECG monitor 32 and can modify the operation of the NIBP determination algorithm based upon the detection of artifacts from the physiological patient monitors, as described.

Although two different types of physiological parameter monitors are illustrated in the embodiment of FIG. 1, it should be understood that the system could utilize other types of monitors while operating within the scope of the present disclosure. As an example, the physiological parameter monitor could be an accelerometer that would be used to directly measure motion of the patient.

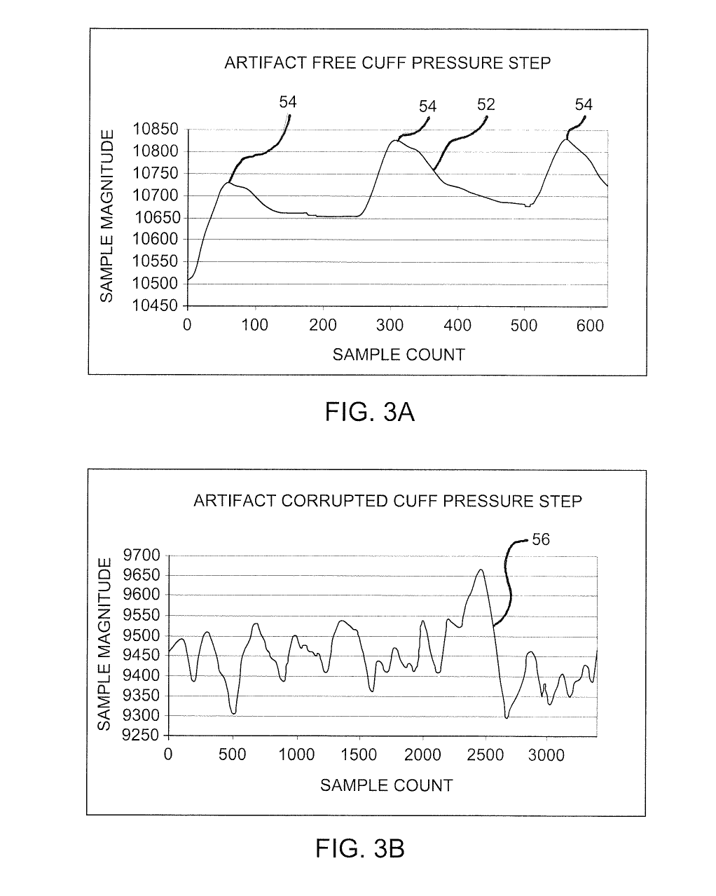

FIG. 3a illustrates the cuff pressure signal 52 received from the blood pressure cuff when the blood pressure cuff is at one of the pressure steps shown in FIG. 2 below the initial inflation pressure during which oscillation pulses are present. As illustrated in FIG. 3a, the cuff pressure signal includes three pressure peaks 54. During operation of the NIBP monitor, the processing unit receives the cuff pressure signal 52 and calculates the amplitude of each of the pressure peaks 54. As described with reference to FIG. 2, the oscillation amplitudes are used to create the oscillometric envelope 42 which has the typical shape shown in FIG. 2. The cuff pressure signal 52 shown in FIG. 3 is relatively artifact-free and is the type of signal required by the NIBP monitoring system to create the oscillometric envelope.

FIG. 3b illustrates the cuff pressure signal 56 at the same cuff pressure step shown in FIG. 3a. The cuff pressure signal 56 shown in FIG. 3 includes a significant amount of artifacts that corrupt the cuff pressure signal at the same pressure step as illustrated in FIG. 3a. As can be understood in the comparison of FIGS. 3a and 3b, the pressure peaks 54 of FIG. 3a are significantly obscured by the artifacts present in the cuff pressure signal. As previously described, the artifacts present in the cuff pressure signal obscure the pressure peaks 54 such that the NIBP monitoring system is unable to calculate oscillometric amplitudes at the pressure step shown in FIG. 3b. When the NIBP monitoring system is operating and receives the cuff pressure signal 56, the NIBP monitoring system is unable to determine oscillometric amplitudes at the individual pressure step and the operating algorithm must then either create an estimate for the oscillometric amplitudes based upon previous measurements or the system will simply return a result indicating that the NIBP monitoring system was unable to calculate the blood pressure of the patient.

If the system terminates operation and returns a result indicating the NIBP monitoring system was unable to calculate the blood pressure, the blood pressure cuff must be re-inflated and the process restarted. Since the determination of the blood pressure of a patient is often uncomfortable and time consuming, it is desirable for the system to attempt to obtain the blood pressure of the patient only at times when significant artifacts are not present.

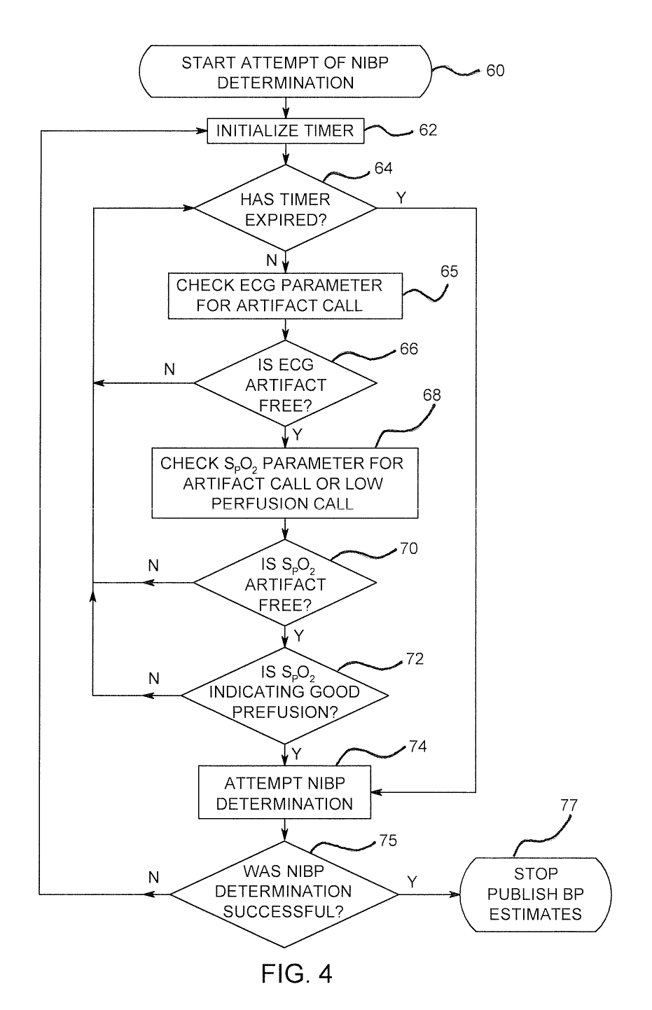

FIG. 4 illustrates the operation of the processing unit of the NIBP monitoring system in controlling the operation of the NIBP monitor as well as determining the blood pressure of the patient after an attempted determination has failed due to artifact corrupting the oscillometric signal obtained from the cuff. After the NIBP determination has begun, the processing unit starts a timer in step 62 and determines whether the timer has expired in step 64. During the initial start-up, the timer will not have expired in step 64. The timer checked in step 64 is used by the processing unit as a timeout device, as will be described in greater detail below.

After checking the timer in step 64, the processing unit will check the signal received from the ECG monitor, as illustrated in step 65. As previously described, the ECG monitor 32 includes an internal operating algorithm that generates a signal indicating whether artifacts or other problems were present in the ECG signal received from the patient that prevented the ECG monitor 32 from performing its normal calculations.

In step 66, the system determines whether the signal received from the ECG monitor indicates that the ECG signal was artifact-free. As previously described, the operating algorithm for the ECG monitor 32 generates a signal that is received by the processing unit 16 and interpreted by the processing unit 16 to determine whether the ECG monitor is generating a signal indicating that the ECG monitor detected artifacts and was unable to process the ECG signal.

If the system determines that the ECG signal is artifact-free in step 66, the processing unit next checks the SpO₂ monitor 31 for both an indication that artifacts are present or whether good perfusion is detected, as indicated in step 68. If the processing unit determines in step 70 that the signal from the SpO₂ monitor 31 indicates that the SpO₂ signal is artifact-free, the processing unit then determines in step 72 whether the SpO₂ monitor 31 is indicating good perfusion. As indicated in FIG. 4, if the processing unit 16 determines that the ECG signal is artifact-free, the SpO₂ signal is artifact-free and that the SpO₂ monitor is indicating good profusion, the system continues to step 74 and begins an attempt to determine the blood pressure of the patient, as will be described in greater detail below.

However, if the processing unit determines in any of the steps 66, 70 or 72 that the physiological parameter monitors, including the SpO₂ monitor 31 and the ECG monitor 32, are detecting artifacts, the processing unit returns to step 64 to determine whether the timer having a predetermined countdown period has timed out. The timer is initialized and started in step 62 at the beginning of the algorithm or subsequent to a determination during which the blood pressure estimates could not be found due to the presence of artifact. The system then returns to step 65 to again check for whether either of the two physiological parameters indicate that artifacts are present.

The timer started in step 62 is checked in step 64. If the timer has expired, the system bypasses the checking steps 65-72 and the system immediately begins to attempt the NIBP determination in step 74. The timer set in step 62 is used to set the maximum amount of time the system continues to check for artifacts in either the ECG signal or the SpO₂ signal. It should be understood that although artifacts may prevent the proper processing of either the ECG signal or the SpO₂ signal, the artifacts detected by these two physiological parameter monitors may not affect the operation of the NIBP monitor. Thus, the timer allows the system to bypass the checking steps after the expiration of the predetermined period such that the processing unit can attempt to complete an NIBP determination.

Once the system attempts to determine the blood pressure of the patient in step 74, the processing unit determines in step 75 whether the NIBP determination method was successful. If the NIBP determination was successful, the processing unit stops the algorithm and publishes the blood pressure estimate in step 77. The blood pressure estimates can be published in many different manners, including the display of the blood pressure estimate on the display unit 30 shown in FIG. 1.

If the processing unit determines in step 75 that the NIBP determination was unsuccessful, the system returns to step 62 and restarts the timer. Once the timer has been restarted, the system checks for a time out in step 64, and returns to step 65 and again checks for whether artifacts are present in either the ECG or SpO₂ signal. It should be understood that in a multi-parameter physiological monitoring system the ECG or SpO₂ may not be enabled at a particular time and will not return a monitoring status signal at all. In this case the ECG or SpO₂ will not provide a mechanism to hold off NIBP determinations. At least one of the other physiological parameters must be capable of delivering a status indicative of the presence of artifact in order for this technique to be effective.

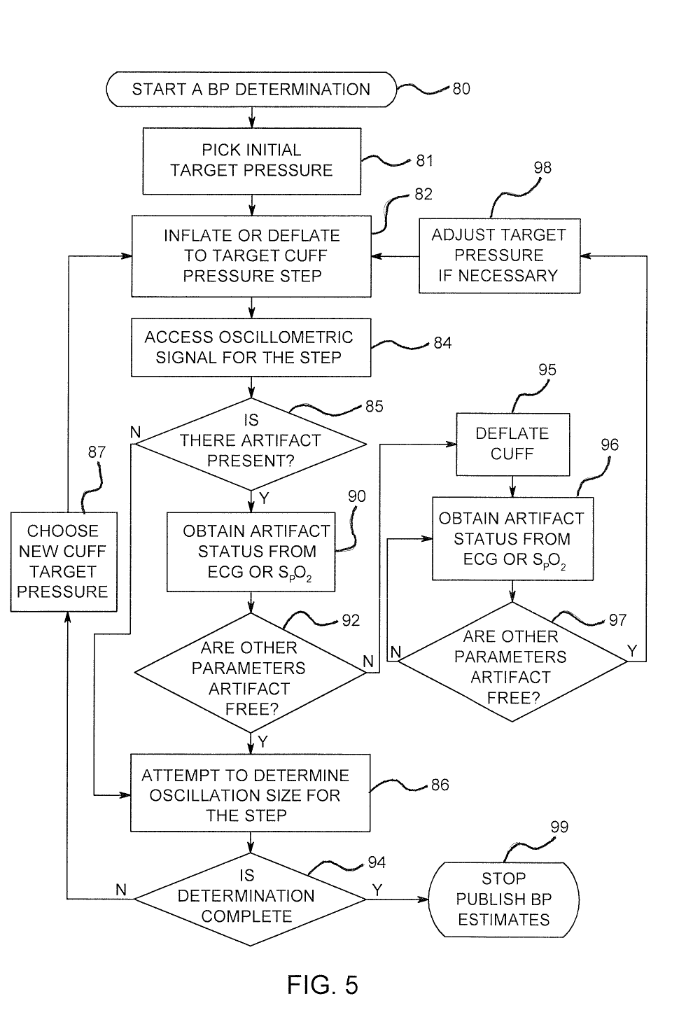

FIG. 5 illustrates the steps carried out by the processing unit 16 in determining the blood pressure of a patient. In general, the steps carried out in FIG. 5 correspond to the NIBP determination step 74 shown in FIG. 4.

Once the processing unit begins the blood pressure determination in step 80, the processing unit 16 pick an initial target pressure for the cuff, as illustrated by step 81, and operates the inflate valve 18 to inflate the blood pressure cuff 12 to the initial inflation target pressure, as illustrated by step 82 in FIG. 5. Once the blood pressure cuff is inflated to the initial target cuff pressure step, the processing unit 16 receives the cuff pressure signal at the current pressure step, as illustrated in step 84. As previously described in FIGS. 3a and 3b, the cuff pressure signal includes pressure peaks 54 when the signal is artifact-free and includes a series of noise fluctuations when the cuff pressure signal is corrupted by artifacts, as illustrated in FIG. 3b. After the cuff pressure signal is obtained, a decision is made as to whether artifacts are present or not, as illustrated by step 85 in FIG. 5. If there is no artifact present, the processing of the cuff pressure signal can proceed normally and obtain estimated complex amplitudes for the oscillometric envelope. However, if artifacts are suspected, then a check is made for the presence of artifact in another physiological parameter.

If no artifacts are present, the processing unit utilizes the operating algorithm in step 86 to attempt to determine the oscillation amplitudes at the specific cuff pressure step. If the processing unit determines in step 85 that the determination of the oscillation amplitudes was unsuccessful, the processing unit 16 checks the signals from the ECG monitor 32 and the SpO₂ monitor 31 in step 90 to determine whether the ECG signal and the SpO₂ signal include artifacts. If the processing unit determines in step 92 that both the ECG signal and the SpO₂ signal are artifact-free, the system returns to step 86 and again attempts to calculate oscillation amplitudes from the cuff pressure signal from the blood pressure cuff.

However, if the processing unit determines in step 92 that the other monitored physiological parameters are not artifact-free, the system proceeds to step 95 to deflate the cuff so as not to harm or cause discomfort for the patient while the artifacts are present. During the deflate period the status of the other physiological parameters are checked for the presence of artifact as illustrated in steps 96 and 97 of FIG. 5. When in the deflate state, a timer could be used to allow the determination to time out if the artifact never diminishes. The system continues to monitor the signals from the two physiological parameter monitors until the artifact is eliminated. If the artifacts are eliminated, the processing unit 16 proceeds to step 98 to determine if there should be an adjustment in the cuff target pressure. Subsequently, the processing unit 16 returns to normal determination progression at step 82.

Referring back to step 86, if the processing unit determines that the oscillation amplitudes are properly calculated and fall within expected results, the system proceeds to step 94 to determine whether the NIBP calculation process is complete. As described with reference to FIG. 2, the process is not complete until the system completely develops the oscillometric envelope 42 shown in FIG. 2. If the oscillometric envelope has not yet been completed, the system returns to step 82 via step 87 where a new cuff pressure is chosen and deflates the blood pressure cuff to the next pressure step. Once deflated, the system continues to monitor the oscillometric amplitude in the manner described.

If the system determines that the NIBP calculation process is complete in step 94, the system proceeds to step 99 and publishes the blood pressure estimates in a known manner, such as on the display 30 shown in FIG. 1.

As can be understood by the above description, the system and method of operating the NIBP monitoring system disclosed increases the likelihood of the successful determination of the blood pressure. The NIBP monitoring system operated in accordance with the present disclosure delays operation of the NIBP monitoring cycle until a physiological parameter monitor indicates that artifacts are not present in the physiological parameters being monitored. By waiting for times when artifacts are not present in the physiological parameters, the NIBP algorithm works more easily to build the oscillometric envelope that includes the information needed to estimate the blood pressure. Further, by waiting for a quiet time without artifacts, the NIBP algorithm will generate a more accurate blood pressure estimate. Additionally, by waiting until a quiet time in which the physiological parameters do not detect artifacts, the NIBP monitoring algorithm will be less likely to carry out the determination process only to end without a blood pressure estimate.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A method of operating a non-invasive blood pressure (NIBP) monitor having a processing unit and a blood pressure cuff positioned on a patient to provide a cuff pressure waveform to the processing unit, the method comprising the steps of:
determining in the processing unit whether artifacts are present in the measurement of a physiological parameter of the patient in a signal received at the processing unit from a physiological parameter monitor; and
adjusting the operation of the NIBP monitor based on the presence of artifacts in the measurement of the physiological parameter.

2. The method of claim 1 wherein the step of adjusting includes delaying the start of the NIBP determination cycle until the artifacts are no longer present in the measurement of the physiological parameter.

3. The method of claim 1 wherein the processing unit determines whether artifacts are present in the measurement of a plurality of distinct physiological parameters from a plurality of physiological parameter monitors.

4. The method of claim 3 wherein the plurality of physiological parameters include an ECG from the patient and the oxygen saturation of the patient.

5. The method of claim 3 wherein the plurality of physiological parameter monitors includes an ECG monitor and an SpO₂ monitor.

6. The method of claim 1 further comprising the steps of:
deflating the blood pressure cuff in a series of pressure steps from an initial inflation pressure;
determining the size of at least one oscillation included in the cuff pressure waveform at each pressure step;
determining in the processing unit whether artifacts are present from the physiological parameter monitor when the size of the at least one oscillation at a current pressure step cannot be determined; and
wherein the step of adjusting includes delaying acquisition of the cuff pressure waveform from the cuff pressure step until the artifacts are no longer present in the signal from the physiological parameter monitor.

7. The method of claim 6 wherein the processing unit determines whether artifacts are present in the measurement of a plurality of distinct physiological parameters from a plurality of physiological parameter monitors.

8. The method of claim 7 wherein the plurality of physiological parameter monitors include an ECG monitor and an SpO₂ monitor.

9. A method of computing a blood pressure of a patient, comprising the steps of:
inflating a blood pressure cuff positioned on the patient to an initial inflation pressure;
deflating the blood pressure cuff in a series of pressure steps from the initial inflation pressure;
determining the size of at least one oscillation amplitude in the processing unit for each pressure step;
determining in the processing unit whether the amplitude determined at each pressure step is consistent with a typical blood pressure determination;
determining in the processing unit whether artifacts are present in the measurement of at least one physiological parameter of the patient from at least one physiological parameter monitor when the determined oscillation amplitude is not consistent with the typical blood pressure determination; and
delaying processing of blood pressure cuff waveform until artifacts are no longer present.

10. The method of claim 9 further comprising the steps of:
determining in the processing unit whether artifacts are present in the measurement of the at least one physiological parameter of the patient from the at least one physiological parameter monitor before inflating the blood pressure cuff; and
delaying the beginning of a blood pressure measurement cycle when artifacts are present.

11. The method of claim 10 wherein the beginning of the blood pressure measurement cycle is delayed until artifacts are no longer present.

12. The method of claim 10 wherein the beginning of the blood pressure measurement cycle is delayed until the artifacts are no longer present or until the expiration of a predetermined delay period.

13. The method of claim 9 wherein the deflation of the blood pressure cuff is delayed until the artifacts are no longer present or until the expiration of a predetermined delay period.

14. The method of claim 9 wherein the processing unit determines whether artifacts are present in the measurement of a plurality of distinct physiological parameters of the patient from a plurality of physiological parameter monitors.

15. The method of claim 14 wherein the plurality of physiological parameter monitors include an ECG monitor and an SpO₂ monitor.

16. The method of claim 10 wherein the processing unit determines whether artifacts are present in the measurement of a plurality of distinct physiological parameters from a plurality of physiological parameter monitors.

17. The method of claim 16 wherein the plurality of physiological parameter monitors includes an ECG monitor and an SpO₂ monitor

18. A non-invasive blood pressure monitoring system, comprising:
a blood pressure cuff that generates a cuff pressure waveform when positioned on a patient;
a processing unit coupled to the blood pressure cuff to receive the cuff pressure waveform from the blood pressure cuff and to inflate the blood pressure cuff to an initial inflation pressure and deflate the blood pressure cuff in a series of pressure steps; and
at least one physiological parameter monitor in communication with the processing unit to deliver a monitoring signal to the processing unit related to a physiological parameter monitored by the physiological parameter monitor, wherein the processing unit is programmed to:
determine whether artifacts are present in the measurement of the physiological parameter of the patient in the monitoring signal received at the processing unit from the physiological parameter monitor; and
adjust the operation of the NIBP monitor based on the presence of artifacts in the physiological parameter.

19. The non-invasive blood pressure monitoring system of claim 18 wherein the at least one physiological parameter monitor includes an ECG monitor and an SpO₂ monitor.

20. The non-invasive blood pressure monitoring system of claim 19 wherein the processing unit delays the inflation of the blood pressure cuff until artifacts are no longer present in the monitoring signal.
